(19) United States
(12) Patent Application Publication
(10) Pub. No.: US 2026/0141739 A1
(43) Pub. Date: May 21, 2026

(54) METHOD AND APPARATUS FOR DOCUMENTATION OF AN OPERATION ON A PATIENT

(71) Applicant: FORBENCAP GmbH, Sonthofen (DE)

(72) Inventor: Robert Ludwig CONLE, Sonthofen (DE)

(21) Appl. No.: 19/390,910

(22) Filed: Nov. 17, 2025

(30) Foreign Application Priority Data
Nov. 21, 2024 (DE) 10 2024 134 270.6

(51) Int. Cl. G06V 20/70; G06V 10/776; G06V 20/52; G16H 15/00

(52) U.S. Cl. CPC G06V 20/70; G06V 10/776; G06V 20/52; G16H 15/00

(57) ABSTRACT

A method for automated documentation of an operation of a patient which may have the steps of: capturing image data of interactions between a surgeon and/or a surgical utensil and a patient using an image capturing unit; processing the captured image data using an image processing algorithm to recognize the interactions in the image data; and generating text and/or speech labels for the recognized interactions using a machine learning label generation model for automated documentation of the surgery using the generated text and/or speech labels.

### CROSS-REFERENCE TO RELATED APPLICATION(S)

This application claims the benefit of German Application No. DE 10 2024 134 270.6, filed Nov. 21, 2024, which is incorporated herein by reference in its entirety.

### TECHNICAL FIELD

The invention relates to a method and a device for the automated documentation of a patient's operation.

### BACKGROUND

The documentation of operations is a crucial part of modern medical processes, as it serves to ensure traceability and quality assurance as well as legal and medical safety. Until now, this documentation has mainly been carried out manually, which is time-consuming and takes up valuable resources of the surgical team. Digital approaches, such as the use of tablets or other input devices, are increasingly being used, but still require considerable human interaction.

With the increasing complexity of operations and the growing pressure to increase efficiency, the need for precise and time-saving documentation is increasing. Reducing the amount of documentation required could allow surgeons to concentrate more on the actual performance of operations and at the same time improve the quality of documentation.

It is a task of the invention to provide a method and/or a device for the automated documentation of a patient's operations, which minimizes the manual effort and increases the precision of the documentation.

### SUMMARY

The problem is solved by a method according to claim 1 and by a device according to claim 10.

According to a preferred aspect, a method for automated documentation of an operation of a patient is proposed. This method comprises the steps of: capturing image data of interactions between a surgeon and/or a surgical utensil and a patient using an image capture unit, processing the captured image data using an image processing algorithm to recognize the interactions in the image data, and generating text and/or speech labels for the recognized interactions using a machine learning label generation model.

The method provides for image data of interactions between a surgeon and/or a surgical utensil and a patient to be captured. This image data preferably documents typical actions during an operation, such as the preparation of a surgical utensil, the preparation of tissue, the placement of an implant and/or the performance of a suture. The image recording unit can take the form of a portable camera, for example integrated into surgical goggles, or as a stationary device located in the operating room. Alternatively, the image acquisition unit could also include depth cameras or multispectral cameras in order to capture additional details of the interactions. Preferably, the image acquisition unit can also be part or a component of existing operating room equipment. For example, an existing image acquisition unit in a microinvasive surgical device can also be used for the present application. Preferably, the image data generated or that can be generated by the image acquisition unit is processed in the manner described above in order to create the automatic surgical documentation.

Furthermore, the captured image data is processed using an image processing algorithm to recognize the interactions contained in the image data. Such an algorithm can, for example, be based on methods such as segmentation, object recognition or motion analysis. Additionally or alternatively, approaches for activity detection, anomaly detection or object tracking could be integrated to identify complex surgical steps or to detect critical situations, such as the risk of a surgical utensil remaining in the patient's body.

In a further step of the process, text and/or speech labels are generated from the recognized interactions. This is done using a machine learning model, preferably a transformer model, which has previously been trained using annotated operation protocols and image data. The labels preferably describe the operation steps performed precisely and in a structured manner, particularly in chronological order, and can be output as text and/or in the form of audio. Alternatively or additionally, other learning models, such as recurrent neural networks (RNNs) or convolutional neural networks (CNNs), could also be used to handle specific tasks such as the analysis of temporal image sequences.

In addition, the procedure could generate a warning message if a detected interaction deviates from a standardized surgical protocol, thus increasing safety during the operation.

The proposed procedure offers numerous technical advantages. It reduces the manual effort required to document operations, giving surgeons and the surgical team more time to perform and monitor operations directly. The use of machine learning models ensures a high level of precision and consistency in the documentation. The automatically generated data can preferably be stored in an audit-proof manner and reviewed as required in order to make the course of the operation traceable and minimize potential risks. The flexibility of the process allows it to be adapted to different surgical environments and/or scenarios, while its scalability supports implementation in facilities of all sizes, from small outpatient surgery centers to large hospitals. In addition, the ability to anonymize data ensures compliance with data protection requirements, especially when processing sensitive patient data.

It is understood that the steps according to the invention and further optional steps do not necessarily have to be carried out in the order shown, but can also be carried out in a different order or in parallel. Further intermediate steps may also be provided. The individual steps may also comprise one or more sub-steps without thereby departing from the scope of the method according to the invention.

The machine learning model preferably generates labels automatically from image data by learning to link visual patterns, movements and contextual information with specific meanings during training. This process is preferably carried out in several interlinked steps. First, the captured image data could be pre-processed to make it consistent and easier for the model to process. This preferably includes adjusting the image size, normalizing colour spaces and removing image noise. For video data, sequences of frames could preferably be extracted in order to analyze temporal sequences, such as movements of surgical utensils or specific hand movements. The machine learning model preferably has a language model, in particular a large language model.

The machine learning model may have a GPT (Generative Pre-trained Transformer), which is based on the Transformer architecture and is particularly well suited for processing text and generating natural-sounding speech. The machine learning model may also have a BERT (Bidirectional Encoder Representations from Transformers), which is also based on the Transformer architecture and is characterized by its ability to bidirectionally analyze contextual relationships in texts. Alternatively, the machine learning model can have a T5 (Text-to-Text Transfer Transformer), which also uses a transformer architecture and specializes in formulating each text processing task as a text-to-text problem.

Furthermore, the machine learning model can have an XLNet, which is based on a transformer architecture with autoregressive and autoencoder-like mechanisms and can better model contextual dependencies. Another possible model could be RoBERTa (Robustly Optimized BERT Approach), an optimized version of BERT that achieves higher performance through more extensive training on larger data sets. The model could also be an ALBERT (A Lite BERT), a lightweight and optimized version of BERT that requires less memory and is faster to train.

The use of these models enables precise and structured documentation of the recognized interactions, which significantly improves the quality and consistency of the surgical protocols.

In addition, the machine learning model can feature an OpenAI Codex, a Transformer-based model specifically trained to process and generate code. For multimodal applications, a CLIP (Contrastive Language-Image Pre-training) model can be used, which is based on a Transformer architecture and combines text and image information to relate visual and linguistic input. Finally, a Transformer-XL could be used, an extended Transformer architecture that can model longer contextual dependencies.

The model preferably extracts visual features from the data. In the first layers of a neural network, for example in Convolutional Neural Networks (CNNs) or Transformer models, basic patterns such as edges, colors or textures can be recognized. Advanced layers preferably abstract these features further and identify more complex structures such as objects or specific actions, such as holding a surgical utensil or placing an implant.

Object recognition and scene analysis algorithms such as YOLO or Mask R-CNN could preferably be used to recognize and segment individual objects in the images or videos. This analysis preferably makes it possible to recognize specific surgical activities such as the application of a scalpel or the insertion of a catheter in their visual context. For dynamic scenes in which movements are crucial, the model could analyze movements across multiple frames. Optical flow algorithms or 3D CNNs could preferably be used to identify activities such as suturing a wound or removing a foreign body.

In order to link the information in a meaningful way, the model preferably interprets the recognized patterns and movements in the context of the operation. Transformer models or recurrent neural networks (RNNs) could preferably be used, as they take into account temporal and spatial relationships. For example, the model could recognize whether the surgeon is preparing a surgical utensil or performing a specific action on the patient. This contextual interpretation is preferably crucial for generating precise labels.

In the final step, the results of object recognition, movement analysis and context interpretation could preferably be compared with previously learned surgical protocols. Based on this analysis, the model could create labels that describe the recognized action or observation, such as “surgical instruments sterilized”, “implant placed” or “incision closed”.

The model can preferably generate these labels automatically because it has been trained on annotated datasets containing images and videos with precise descriptions. During the training process, it could learn how specific visual patterns, such as the movement of a scalpel carrier or the position of a clamp, correlate with specific surgical activities. In addition, Transformer models could preferably combine information from multiple sources, such as image data, movement patterns and environmental information, to provide a contextual interpretation of the scene.

A practical example of automatic label generation could preferably be the placement of an implant. A camera could record how the surgeon places the implant in the intended position. The model could recognize the surgeon, the implant and the movement sequence and preferably generate the label “implant successfully placed”. In another scenario, the model could analyze the movements of the surgeon and an assistant during a suture and preferably generate the label “Wound closure performed”. Also, when using image-guided techniques, such as navigation of a catheter, the model could automatically recognize the utensil, the position of the patient and the interaction to preferably generate the label “Catheter inserted”.

In a further preferred aspect, a device for the automated documentation of operations on a patient is proposed, the device having an evaluation and computing device which is designed to perform the following steps: acquiring image data of interactions between a surgeon and/or a surgical utensil and a patient using an image acquisition unit; processing the acquired image data using an image processing algorithm for recognizing the interactions in the image data; and generating text and/or speech labels for the recognized interactions using a machine learning model, in particular a transformer model.

The statements made for the method apply accordingly to the device. It is understood that linguistic modifications of features formulated in terms of the method can be reformulated for the device in accordance with standard linguistic practice, without such formulations having to be explicitly listed here.

In a further aspect, it is proposed that the method comprises or utilizes an image capturing unit comprising a camera and/or a portable image capturing device, such as surgical goggles or another portable camera system, or accessing image data from an image capturing unit already in use.

The image acquisition unit preferably enables the acquisition of visual data of the interactions between a surgeon and/or a surgical utensil and a patient during an operation. The camera can be installed stationary in the operating room, for example on an operating light or on the operating table, or it can be used as a mobile device. Portable devices such as surgical goggles increase the flexibility of the surgeon and can capture context-relevant data such as the direction of gaze. Alternatively or additionally, wearable devices such as body cameras attached directly to the surgeon or surgical utensil could be used. These features preferably facilitate integration into the operating room workflow without interfering with the performance of the surgery. The use of portable imaging units enables the acquisition of image data in close proximity to the surgical action, which increases the precision of the documentation. Portable devices minimize space requirements and maximize mobility, while stationary units ensure continuous and comprehensive recording of the surgical procedure.

In a further aspect, it is proposed that the method comprises an image acquisition unit which is installed in a stationary or movable manner in an operating room and/or is arranged on a body of the surgeon or on a surgical utensil.

The arrangement of the image acquisition unit preferably determines the perspective and range of the captured data. Stationary units, such as cameras mounted on the ceiling or a surgical light, enable comprehensive monitoring of the surgical area. Movable image capture units, such as portable cameras or devices attached to surgical instruments, can preferably be flexibly positioned to capture specific details of interactions. Alternatively, sensors could be attached directly to the surgical utensils or the surgeon's body to ensure personalized capture. Stationary units provide permanent, room-wide coverage, while wearable devices document detailed data up close. Body-mounted cameras provide individual perspectives and reduce the influence of environmental elements. The versatility of the positioning options preferably increases the functionality of the system in different operating environments. Movable image acquisition units improve usability in complex or changing surgical scenarios.

In a further aspect, it is proposed that the image processing algorithm comprises image data segmentation and/or object detection and/or motion analysis and/or activity detection and/or anomaly detection and/or object tracking and/or action classification and/or multimodal image processing and/or feature extraction.

The image processing algorithm preferably analyzes the captured image data and extracts specific information. Segmentation preferably divides the image into relevant areas, while object recognition identifies specific surgical utensils, such as scalpels or clamps. Motion and activity detection are preferably used to analyze dynamic surgical activities, such as cutting, suturing or placing an implant. Anomaly detection could be used to identify critical situations such as the risk of a utensil remaining in the patient. Multimodal image processing preferably combines different types of data, such as RGB and depth data, to enable a more comprehensive analysis of the surgery. These algorithms ensure precise recognition of surgical activities and increase the reliability of documentation. The integration of several algorithms preferably offers high flexibility and adaptability to different surgical scenarios.

In a further aspect, it is proposed that the image processing algorithm comprises a machine learning model for image processing, in particular a convolutional neural network (CNN).

Convolutional neural networks (CNNs) are particularly suitable for image processing and can efficiently recognize patterns and objects in image data. Alternatively or in addition, transformer models or hybrid approaches can be used to analyze complex temporal and spatial dependencies. These models increase the accuracy and speed of image processing and promote the automation of documentation.

In general, the image processing algorithm may comprise a machine learning model and/or a statistical model and/or an analytical model, in particular a hybrid model comprising several of the aforementioned models in combination.

In a further aspect, it is proposed that the machine label generation learning model is trained or at least fine-tuned using standardized operation protocols, the operation history and associated image data.

Training the model on specific and/or predetermined operation protocols preferably enables application-specific customization. Alternatively, publicly available or synthetically generated data could be used to initialize the model. Training on specific data preferably increases accuracy and context sensitivity. Fine-tuning allows for personalized and context-dependent documentation of the operation.

In a further aspect, it is proposed that the generated text and/or speech labels are output as an audio and/or text file, in particular an editable audio and/or text file.

The output in various formats enables easy integration into existing systems, such as electronic surgical protocols or hospital information systems (HIS). Alternatively, labels can be visualized in dashboards to provide the surgical team with a real-time overview of the documented steps. Such flexibility in the output preferably promotes the usability and adaptability of the system. The editable output preferably facilitates corrections and/or adaptations to individual requirements.

In a further aspect, it is proposed that erroneous labels are identified in a revision of the operation protocols and subsequently used to retrain the machine label generation learning model and/or the image processing algorithm.

The continuous improvement of the model through faulty labels preferably promotes the ability to learn. Alternatively, external sources of feedback, such as comments from operators or protocol reviewers, could be included. Post-training increases long-term accuracy and robustness. The system remains adaptive and adapts to changing conditions or new protocols.

In a further aspect, it is proposed that a computer program product comprises instructions which, when the program is executed by a computer, cause the computer to perform the steps of the present method according to any embodiment.

The computer program product forms the basis for implementing the procedures. The availability as a software product facilitates distribution and integration into existing OR systems. Simple use on existing hardware is made possible.

A computer program that implements the steps of a procedure for the automated documentation of operations can preferably have a modular structure and consist of several components that perform specific tasks. For example, it could consist of the following modules:

An input module is preferably used to integrate the image acquisition unit, which is either stationary, portable or mobile. This input module preferably controls the acquisition of the image data, synchronizes the images with other data sets (e.g. time stamps or patient information) if required and/or prepares the image data for processing. In addition, this input module can apply filters to optimize image quality and integrate privacy-friendly techniques such as anonymization or blurring of sensitive data.

An image processing module preferably analyzes the captured image data using one or more image processing algorithms. Neural networks such as convolutional neural networks (CNNs) or vision transformers (ViTs) could be used for object recognition, segmentation, motion analysis or activity recognition. This image processing module is preferably designed to extract relevant information such as the surgeon's actions, the use of surgical instruments or the progress of the operation.

A label generation module preferably processes the results of the image processing module and generates text and/or speech labels. This label generation module preferably uses a machine learning model, such as a transformer model, which has been trained on annotated operation logs and image data. The labels are preferably generated in a structured form and can be formatted as text or audio output as required. Furthermore, the label generation module can include a feedback component that identifies faulty labels and uses them to optimize the machine learning model.

An output module preferably takes care of the storage and output of the generated labels. It could provide the labels in various formats, such as editable text files, audio formats or in the form of a visual user interface, such as a dashboard displaying real-time updates. This output module can also provide an interface for integration with external systems, such as electronic surgical records or hospital information systems (HIS).

A training and adaptation module preferably enables the further development and fine-tuning of the models used. It could perform retraining based on new data or faulty labels and thus continuously improve the precision and robustness of the system. It could work both online (during use) and offline (on prepared data sets) to integrate new surgical techniques or protocol requirements.

A security module preferably ensures that data protection requirements are met. It could use encryption techniques to secure the stored data and restrict access to the data to authorized users. This module can also integrate mechanisms for anonymizing sensitive information such as faces or identifying features of the patient in order to comply with data protection regulations.

In a further aspect, it is proposed that the automated documentation method is further adapted to generate a warning message using the generated text and/or voice labels when an interaction deviates from a surgical protocol and/or there is a risk that a surgical utensil may remain in a patient's body. This function serves to increase safety during an operation and minimize human error.

To detect deviations, the procedure compares the generated labels with a predefined surgical protocol. The surgical protocol contains, for example, a standardized, step-by-step description of the activities required for the respective procedure, including the sequence and expected time periods for certain steps. The system uses this information to check whether the actions documented during the operation comply with the protocol. Deviations in time sequence, content execution or completeness can be detected. If, for example, a step is skipped or carried out in an unintended sequence, the system generates a warning message indicating the deviation.

The system also monitors the handling of surgical utensils. To do this, the machine learning model analyzes both the visually captured image data and the generated labels to ensure that each surgical utensil used is correctly identified and logged. At the end of the operation, the system can automatically check whether all utensils used have been returned as expected. If a registered surgical utensil is missing, a warning message is issued to alert the surgical team that a utensil may remain in the patient's body. This can be done, for example, by comparing the initially recorded list of instruments used with the instruments returned at the end.

The alerts are preferably issued in real time and can be provided visually via a display unit or audibly as a voice message. Visual alerts could, for example, be displayed on a monitor in the operating room, while audible alerts are provided to the surgical team via loudspeakers or portable devices. The alerts are preferably precise and give specific indications of the nature of the deviation or potential risk, for example: “Step 4 of the protocol has been skipped” or “Instrument X not returned-review required.”

For risk detection, the system preferably uses multimodal data fusion, which combines visual, textual and temporal information. For example, by continuously analyzing interactions and movements, the system could detect if a surgical utensil is in an unusual position or remains motionless for an unexpectedly long period of time. Such anomalies can be an indicator that an instrument has inadvertently remained in the patient's body. In other words, if a number of attached utensils does not match a number of returned utensils, a warning signal is preferably generated.

The flexibility of the system makes it possible to adapt the warning messages to specific requirements or scenarios. In emergencies, for example, the system can adjust the priority of the alerts and highlight particularly critical situations. In addition, the system can be continuously improved through machine learning by learning from past alerts and their resolutions in order to further increase the precision and relevance of the alerts.

The integration of this function into the procedure contributes significantly to patient safety by reducing human error and supporting the surgical team at critical moments. The ability to detect potentially life-threatening situations at an early stage minimizes the risk of complications and contributes to higher quality and reliability in the performance of operations.

For example, the method is designed to generate specific alerts that recognize critical situations such as forgetting a swab in the patient's body. For example, the system could issue a warning message if it is detected that the surgical suture has already started while not all surgical utensils inserted into the body, such as swabs, have been removed. Such a warning message could read: “Warning: swab not removed-check required before suturing is completed.” This function can prevent potentially serious secondary diseases, such as internal infections and inflammation, at an early stage.

For example, the procedure continuously counts the number of surgical utensils inserted into the body, such as swabs, sponges and/or instruments. This number is monitored throughout the operation and compared with the number of utensils removed, for example. If a discrepancy is detected, the system generates a warning and prompts the surgical team to carry out a check. This is done in real time so that appropriate action can be taken before the operation is continued or completed.

The function is based on a combination of visual object recognition and text labels that document the surgical staff's interactions with the utensils. For example, the machine learning model could recognize when a swab is inserted into the body and automatically record this action. It also records when the swab leaves the body again. If the number of swabs removed does not match the number of swabs inserted, this is detected immediately.

This automated monitoring minimizes the risk of human error, for example, and significantly increases patient safety. The system also supports the surgical team by performing routine tasks such as counting utensils and generating precise alerts. This reduces errors, reduces the team's workload and ensures greater precision and efficiency during the operation.

In a further aspect, it is proposed that a computer-readable data carrier stores the present computer program product.

The data carrier is preferably used for long-term storage and distribution of the program. Storage on data carriers preferably ensures portability and enables flexible distribution and backup. A typical data carrier could be a physical medium such as a CD, DVD or Blu-ray disc on which the program is permanently stored. Alternatively, a flash-based storage medium such as a USB stick or SSD could be used, which offers a larger storage capacity and easier handling.

Another approach would be a cloud-based data carrier, where the program data is stored on a remote server and can be accessed via the internet. Such solutions are particularly suitable for scenarios in which updates and shared access to the program are required. In all cases, the data carrier could provide additional security mechanisms such as password protection or encryption to prevent unauthorized access.

The data carrier can also be designed to be directly compatible with existing systems, such as hospital information systems, and enable seamless integration. This promotes easy distribution and scalability of the program in different operating environments.

The training of the machine learning model for the present method can preferably take place in several phases in order to flexibly meet the requirements of the automated operation documentation. First, a basic model of the machine learning model is preferably provided, which is trained on general data before being fine-tuned using specific operation data. This process preferably includes the phases of data collection, data preparation, model training, validation and optimization to ensure high accuracy and robustness.

The training data for the machine learning model may preferably come from a variety of sources, such as video and image recordings of real surgeries, annotated surgical protocols or synthetically generated data. This data could include different surgical activities, such as inserting an implant, suturing a wound or removing a foreign body, and could be supplemented by metadata such as timestamps, environmental features or sensor information. The training data can preferably be in formats such as MP4 or AVI for video data, JPEG or PNG for images and CSV or JSON for associated annotations. Annotations could contain labels that assign semantic meanings to the data, such as “implant inserted” or “wound closure performed”.

The training data is preferably processed by the machine learning model by means of pre-processing, which includes normalization of the image data (e.g. adjustment of resolution and colour values), extraction of relevant features (e.g. movement patterns or object contours) and segmentation of the scenes. The annotated labels are preferably used as target values for training the model. During the training process, the model gradually abstracts the features in order to learn the relationships between the inputs (image data) and the outputs (labels).

A foundation model approach could preferably be used to increase efficiency. A large pre-trained model is used, preferably trained on extensive, general data sets such as large video or image databases. This foundation model preferably has a broad understanding of general visual and semantic patterns and reduces the need for extensive, operation-specific data. Subsequently, the foundation model is preferably fine-tuned with domain-specific surgical data, such as annotated videos from operating rooms, to cover the specific requirements of surgical documentation.

An application example of the present method could preferably be used in a hospital for the documentation of surgical procedures. In such a scenario, a stationary camera installed in the operating room could preferably record the interactions between the surgeon and the patient. The system preferably records scenes such as the handing of surgical instruments, the placement of implants or the suturing of a wound. The camera could preferably transmit the data to the image processing module, which analyzes the scenes in real time and recognizes relevant movement patterns. A machine learning model, which has preferably been specially trained on surgical data, could then generate text and/or voice labels such as “implant successfully placed” or “wound closure completed”.

The generated labels could be automatically integrated into the hospital's electronic surgical protocol. At the same time, a dashboard interface could allow the surgical team to review the documentation in real time and correct it if necessary. Incorrect labels could preferably be adjusted manually, with these adjustments feeding into the feedback loop of the machine learning model to continuously optimize the system. This example shows how the process can be used efficiently to reduce documentation effort, increase accuracy and reduce the burden on medical staff.

The machine learning model can be further enhanced by the use of additional audio data, preferably captured using a microphone, as this provides an additional dimension of contextual information. Audio data could preferably include speech, ambient noise and/or specific acoustic events such as the clinking of surgical instruments, the opening of packages and/or the sound of medical devices. This information can preferably be captured by a microphone that is either integrated into the image capture unit, such as a stationary camera in the operating room, or used as a separate device. The synchronization of audio and image data could preferably establish temporal correspondences between visual and acoustic signals. For example, the machine learning model could recognize that a surgical staple is being applied by hearing a stapling sound in combination with a visual action.

Speech recognition and/or analysis could preferably be integrated into the system to transcribe spoken words or phrases during an operation. This can preferably provide cues to the action being performed, such as when the surgeon says, “Apply clamp.” Such linguistic cues could complement the visual analysis and assist the model in correctly interpreting the action. In addition, ambient sounds such as the beeping of a monitor, the hissing of an oxygen machine or the clicking of an instrument could be analyzed. These sounds could preferably serve as indicators for certain surgical steps, such as the placement of an endotracheal tube or the opening of a suture.

The combination of visual and audio data through multimodal data fusion can preferably enable a holistic analysis of operations. Transformer models or other specialized architectures for multimodal data could preferably be used to merge visual and audio information. This fusion preferably allows the model to supplement unclear information from one data stream with the other. Training data could preferably be augmented with annotated audio clips containing, for example, typical sounds and speech patterns during operations. This sensitizes the model to acoustic variations such as different pitches and/or dialects.

A practical example shows the advantages of this integration. Suppose a surgeon fixes an implant. The visual model could preferably recognize the surgeon and the implant, but might not be able to clearly classify the specific action. By integrating audio data, the model could hear the sound of screwing and the surgeon saying “implant fixed”. This information allows the model to generate the label “implant fixed” with high precision.

This integration of audio data could make the machine learning model more accurate and flexible, especially in situations where the view of the action is limited and/or visual data alone is not sufficient. The combination of visual and acoustic signals improves contextualization and enables faster, more reliable and more comprehensive automatic documentation of operations.

In a further aspect, data security and patient anonymity can preferably be ensured by a number of technical measures. The captured image data of interactions during the operation can preferably be anonymized before processing by the image processing algorithm. Methods such as facial recognition and masking could be used here to automatically recognize faces and/or other identifiable features and make them unrecognizable by blurring, pixelation or complete coverage.

To prevent unauthorized access, the image data could be secured by end-to-end encryption during transmission from the image acquisition unit to the evaluation and computing unit. An encryption method such as AES-256 could preferably be used to ensure that only authorized systems can decrypt and further process the data. In addition, pseudonymized data structures could be used in which personal identifiers, such as the patient's name, are replaced by unique, untraceable codes.

The data could be stored on local, protected servers that are physically and digitally secured against attacks. Alternatively, edge computing solutions could be used in which the data is processed and anonymized on the image acquisition unit or in a local unit so that no sensitive data has to be transferred to external networks.

To ensure data security, access control systems could be implemented that only allow authorized persons access to certain data areas. These systems could be based on two-factor authentication (2FA) and/or biometric procedures. In addition, all data operations could be logged by an audit logging system in order to identify and, if necessary, block suspicious access attempts.

Finally, a privacy-oriented approach could be complemented by the use of differential privacy, where noise signals are added to the data to prevent inferences about individual patients while retaining the useful information for processing. These technical measures could ensure secure and anonymized processing of the data in the context of the claimed procedure.

### BRIEF DESCRIPTION OF DRAWINGS

The accompanying drawings are intended to provide a further understanding of embodiments of the invention. They illustrate embodiments and, in connection with the description, serve to explain principles and concepts of the invention.

Other embodiments and many of the advantages mentioned are shown in the drawings. The elements shown in the drawings are not necessarily shown to scale in relation to each other.

FIG. 1 shows a schematic flow diagram of an embodiment of the process.

FIG. 2 shows a schematic view of a block diagram of the present device.

FIG. 3 shows a schematic view of a block diagram of the present device.

### DETAILED DESCRIPTION

In the figures in the drawings, identical reference signs denote identical or functionally identical elements, parts or components, unless otherwise indicated.

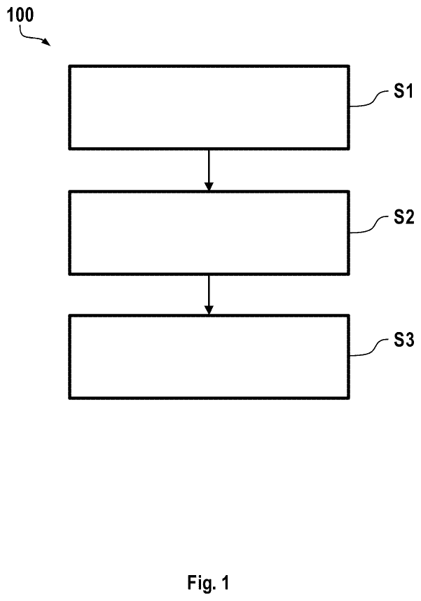

FIG. 1 shows a schematic flow diagram of an embodiment of an existing method for the automated documentation of a patient's operations.

In any embodiment, the method can be carried out at least in part by a device 100, which for this purpose can comprise several components not shown in more detail, for example one or more provision devices and/or at least one evaluation and computing device. It is understood that the provision device may be formed together with the evaluation and computing device, or may be different from the latter. Furthermore, the device 100, which may be part of a system, may comprise a storage device and/or an output device and/or a display device and/or an input device.

The computer-implemented method comprises at least the following steps:

In a step S1, image data of interactions between a surgeon and/or a surgical utensil and a patient is captured using an image capture unit.

In a step S2, the captured image data is processed using an image processing algorithm to recognize the interactions in the image data.

In a step S3, text and/or speech labels are generated for the recognized interactions using a machine learning label generation model for automated documentation of the operation using the generated text and/or speech labels.

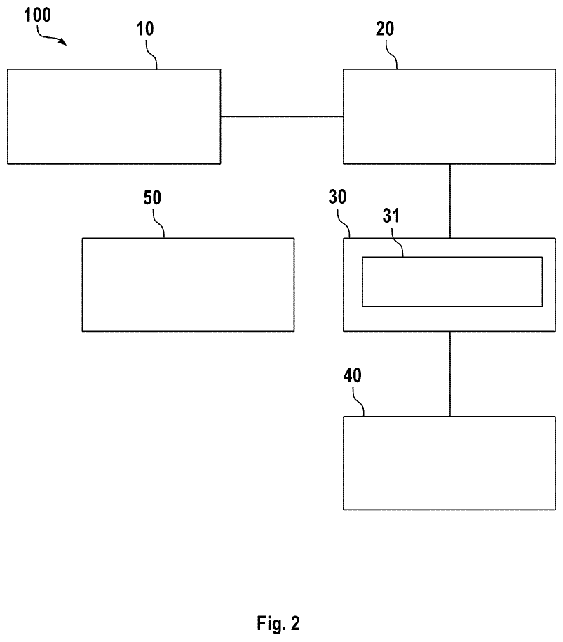

FIG. 2 shows a schematic representation of the architecture of the device 100 for automated documentation of operations. The device 100 includes an image capture unit 10 that captures visual data of interactions 130 between a surgeon 110 and a patient 120. The image capture unit 10 may include various embodiments, such as a stationary installed camera, a wearable camera or a camera integrated into (smart) glasses. The image acquisition unit 10 may also be arranged on a surgical utensil 110 or on the surgeon's head. The captured data is preferably forwarded to an evaluation and computing unit 20 via an interface.

The evaluation and computing unit 20 analyzes the image data and preferably comprises various modules. First, the data is processed by an image processing module 25, which extracts visual features, such as the position of the surgeon 110, the patient 120 and any surgical utensils. The processed image data is then analyzed in the label generation module 30, which automatically generates text and/or speech labels using a machine learning model 31. These labels describe the identified surgical activities, such as “incision performed” or “suture applied”.

The label generation module 30 preferably forwards the generated labels to an output module 40, which preferably provides the output in different formats. The output module 40 preferably enables the generation of text documents that can be integrated directly into electronic patient records and/or the output of voice information to support the surgeon. A processing unit 50, in particular a central processing unit, preferably controls the entire data flow between the individual modules and ensures that the data is processed and forwarded consistently. The architecture enables automated, precise and efficient documentation of operations.

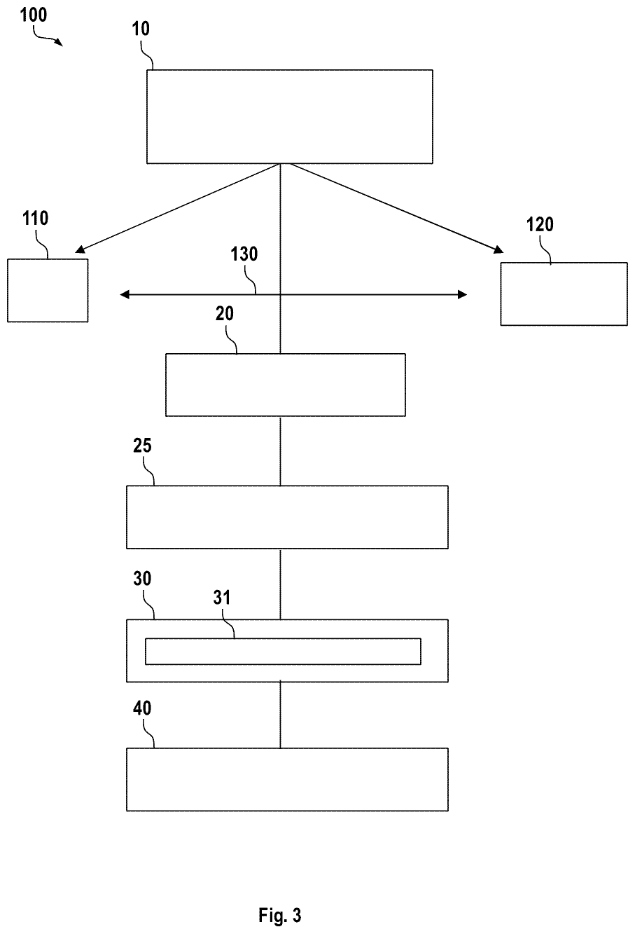

FIG. 3 shows the spatial arrangement of the individual system components in a typical surgical environment. In the center of the illustration, a patient 120 can be seen schematically, who is being operated on by a surgeon 110. The interactions 130 between the surgeon 110 and the patient 120 are captured by an image acquisition unit 10. This image recording unit 10 can be designed as a portable component, for example in the form of smart glasses, or as a stationary camera installed in the operating room.

The image recording unit 10 records the visual data of the interactions 130 and preferably synchronizes it with additional information, such as movement or environmental data. The recorded data is transmitted to the evaluation and computing unit 20, which analyzes, for example, movements, objects and/or actions. For example, it can be recognized whether the surgeon 110 performs an incision, places an implant and/or uses medical devices.

After analyzing the interactions 130 in the evaluation and calculation unit 20, the results are preferably forwarded to the label generation module 30. The label generation module 30 uses at least one machine learning model or a model composition of several machine and/or statistical and/or analytical models to translate the data into, in particular, meaningful labels for documenting the operation. These labels could be, for example, “implant placed”, “incision closed” or “instrument removed”. The labels are preferably created automatically in the context of the operation and transmitted to the output module 40. The output module 40 then preferably outputs the documentation of the operation in a structured form using the generated labels. Preferably, general context information and/or template information on the structure and/or type of documentation can also be generated on the basis of language processing and/or using a language model. This output takes place either as a text document, which is stored in a digital patient file, for example, and/or as a voice file that provides auditory support for the surgeon.

### List of reference symbols

10 Image acquisition unit
20 Evaluation and calculation unit
25 Image processing module
30 Label generation module
31 Machine learning model
40 Output module
50 Process unit
100 Device
110 Surgeon and/or surgical instruments
120 Patient
130 interactions
S1 Step 1 (capturing image data)
S2 Step 2 (Processing image data)
S3 Step 3 (Generating labels)

1. A method for automated documentation of an operation on a patient, comprising:
acquiring image data of interactions between a surgeon and/or a surgical utensil and the patient using an image acquisition unit;
processing the captured image data using an image processing algorithm configured to recognize the interactions in the image data; and
generating text and/or speech labels related to the recognized interactions using a machine learning label generation model, wherein the text and/or speech labels document the operation.

2. The method of claim 1, wherein the image acquisition unit comprises a camera configured for image and/or video recording, and wherein the image acquisition unit is arranged in a room where the patient is operated on, in an operating light, on the body of the surgeon, or on a surgical utensil.

3. The method of claim 1, wherein the image processing algorithm comprises one or more of the following: image data segmentation, object recognition, motion analysis, activity recognition, face recognition, anomaly detection, object tracking, action classification, multimodal image processing, or feature extraction.

4. The method of claim 1, wherein the image processing algorithm comprises a machine learning model for image processing.

5. The method of claim 1, wherein the machine learning label generation model is trained or fine-tuned using standardized surgical protocols and associated image data.

6. The method of claim 1, wherein the generated text and/or speech labels are output as an audio file, a text file, or both.

7. The method of claim 1, further comprising identifying erroneous labels during a revision of the surgical documentation and using the identified erroneous labels to retrain the machine learning label generation model or the image processing algorithm.

8. The method of claim 1, further comprising generating a warning message using the text and/or speech labels if an interaction deviates from a surgical protocol or if there is a risk that a surgical utensil may remain in the body of the patient.

9. A computer program product comprising instructions which, when executed by a computer, cause the computer to perform the method of claim 1.

10. A device for automated documentation of an operation on a patient, comprising:
an image acquisition unit configured to acquire image data of interactions between a surgeon and/or a surgical utensil and the patient;
an evaluation and computing device configured to process the captured image data using an image processing algorithm to recognize the interactions in the image data; and
a machine learning model configured to generate text and/or speech labels for the recognized interactions, wherein the text and/or speech labels document the operation.
